(19) United States
(12) Patent Application Publication
(10) Pub. No.: US 2012/0226095 A1
(43) Pub. Date: Sep. 6, 2012

(54) Personal Item with Energy Enhancement Arrangement

(76) Inventor: Khelin J. YOUNG, Rancho Cucamonga, CA (US)

(21) Appl. No.: 13/404,253

(22) Filed: Feb. 24, 2012

Related U.S. Application Data
(63) Continuation-in-part of application No. 13/035,461, filed on Feb. 25, 2011.

(51) Int. Cl. A61M 21/02

(52) U.S. Cl. 600/15

(57) ABSTRACT

A personal item includes a body being carried by a user and a plurality of different energy elements secured in the body. The energy elements are selected from a group consisting of hologram element, germanium, titanium, and an element carrying negative ions for generating a particular magnetic force which helps rebalance and restore natural energy pathways of the user. So, when the user carries the body, the energy elements are held close enough to the user in order to react positively with the user's energy field. Therefore, the personal item of the present invention is able to improve the blood circulation, recovery time, flexibility, strength, balance, healing and energy as well as contributing to an overall sense of health and well being among its users.

### CROSS REFERENCE TO RELATED APPLICATIONS

This application is a continuation-in-part of U.S. patent application Ser. No. 13/035,461, entitled “Personal Item with Energy Enhancement Arrangement”, filed Feb. 25, 2011.

### BACKGROUND

1. Field of the Invention

The present invention relates to personal items. More particularly, the present invention relates to a personal item with an energy enhancement arrangement that produces a range of frequencies found in nature to react positively with the user's body energy field.

2. Discussion of the Related Art

Holographic disk is commonly recognized as an energized element to improve the user's balance, improve synaptic function in the brain, and enhance muscle response. Since human body generates a magnetic field which can get out of balance, the holographic disk can generate an electrical frequency to restore the magnetic balance of the human body. But, the most vital factor of the holographic disk must be close enough to the user's body in order to “receive” the electrical frequency from the holographic disk.

People often carry personal items such as key chain, purse, wallet, and mobile phone or wear them such as bracelet, jewelry, watches when they are traveling. The personal items can be considered as the most intimate belongings because they are placed close to the user's body. Therefore, holographic disk can be placed on the personal items, such as hologram bracelet, for the user's body to communicate with the holographic disk.

The major drawback of the hologram bracelet is that the holographic disk is not well protected. Generally speaking, the holographic disk is a film coated or printed on the bracelet surface. Therefore, any unintentional contact or scratch will damage the holographic disk. The damaged holographic disk will lose or degrade the magnetic field generation.

### BRIEF SUMMARY OF THE INVENTION

The present invention overcomes the above mentioned drawbacks and limitation by configuring the personal item with the energy enhancement arrangement.

The primary objective of the present invention is that the personal item with an energy enhancement arrangement can produce a range of frequencies found in nature to react positively with the user's body energy field.

The personal item comprises a body to carry the energy enhancement arrangement. The body can be carried or worn by the user in order to have close contact with the energy enhancement arrangement. The body can be bracelet, jewelry, watches, clothing, headwear, footwear, eyewear, key chain, purse, wallet, belt, or sporting goods.

The energy enhancement arrangement comprises a plurality of different energy elements, wherein each of the energy elements is adapted for generating a particular magnetic force which helps rebalance and restore natural energy pathways of the user.

In the preferred embodiment, the energy elements are hologram element, germanium, and titanium. Particularly, two or more, preferably three, of the energy elements are held by the body. Therefore, when the user carries the body, the energy elements are held close enough to the user in order to react positively with the user's energy field.

Another objective of the present invention is that the energy elements are embedded in the body in order to well protect the energy elements. All the energy elements at the body are irreplaceable.

Another objective of the present invention is that the hologram element is sealed and enclosed within an indention of the body and is covered by the cover. Therefore, the hologram element can be kept in tact to prolong the life span thereof.

Another objective of the present invention is that all the energy elements are nature and compatible with the human body. Therefore, the personal item is safe to be carried or worn by the user to react with the energy elements.

For a more complete understanding of the present invention with its objectives and distinctive features and advantages, reference is now made to the following specification and to the accompanying drawings.

### BRIEF DESCRIPTION OF THE DRAWING(S)

FIG. 1 is a schematic view of the personal item with the energy enhancement arrangement in accordance with the present invention, illustrating the body being configured as a bracelet.

FIG. 2 is a sectional view of the personal item with the energy enhancement arrangement in accordance with the present invention, illustrating the energy elements embedded in the body.

FIG. 3 is a schematic view of the personal item with the energy enhancement arrangement in accordance with the present invention, illustrating an alternative configuration as a pair of spectacles.

FIGS. 4A and 4B are schematic view of the personal item with the energy enhancement arrangement in accordance with the present invention, illustrating an alternative configuration as a fitness gear.

FIG. 5 illustrates the application of the energy enhancement arrangement with different kinds of personal item in accordance with the present invention.

FIG. 6 shows another modification of the personal item with the energy enhancement arrangement in accordance with the present invention, illustrating an alternative configuration as a pendant necklace.

### DETAILED DESCRIPTION OF THE INVENTION

In accordance with a preferred embodiment of the present invention, the present invention provides a personal item which can produce a range of frequencies found in nature to react positively with the user's body energy field, wherein when the user carries the personal item of the present invention, it is able to improve the blood circulation, recovery time, flexibility, strength, balance, healing and energy as well as contributing to an overall sense of health and well being among its users. With regard to the present invention, the personal item comprises an energy enhancement arrangement 10 and a body 20.

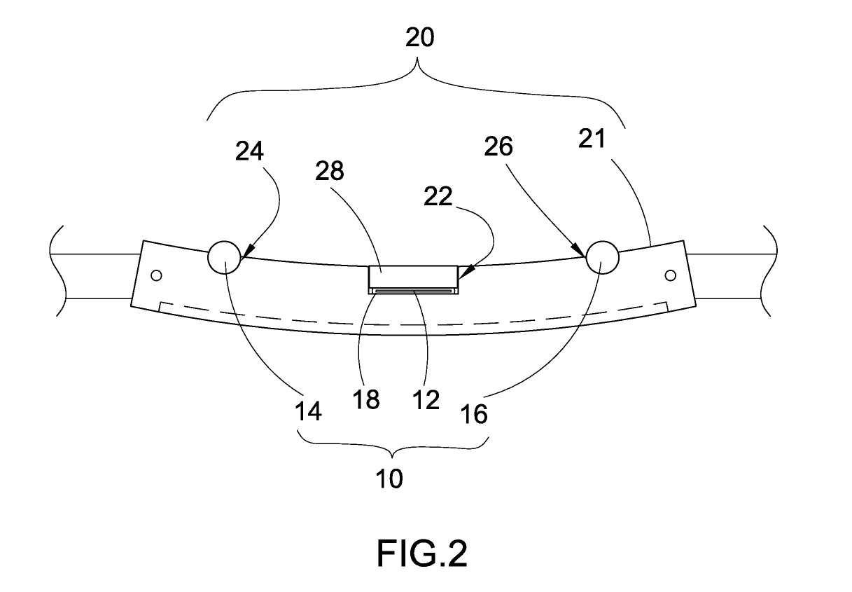

The energy enhancement arrangement 10 comprises a plurality of different energy elements 12, 14, and 16 selected from a group consisting of hologram element, germanium, and titanium. Hologram element, germanium, and titanium are assigned with a reference character 12, 14 and 16 in the figures respectively. Each of the energy elements is adapted for generating a particular magnetic force which helps rebalance and restore natural energy pathways of the user. It is preferred that hologram element 12 is sealed within a waterproof sleeve 18 for protection as in FIG. 2.

The body 20 is arranged for being carried by the user, wherein at least two of the different energy elements 12, 14, and 16, preferably all three different energy elements, are held by the body 20, whereby, when the user carries the body 20, the energy elements 12, 14, and 16 are held close enough to the user in order to react positively with the user's energy field.

In accordance with the preferred embodiment of the present invention, the energy elements 12, 14, and 16 are embedded in the body 20. The body 20 has a plurality of spaced apart indentions 22, 24, 26 indent on the surface of the body 20, wherein the energy elements 12, 14, 16 are received in the indentions 22, 24, 26 respectively. It is noted that the hologram element 12 is sealed within the waterproof sleeve 18 and is received in the respective indentions 22.

Germanium 14 is configured with a size and shape fitting in the respective indention 24. Titanium 16 is also configured with a size and shape fitting in the respective indention 26. It is preferred the size and shape of germanium 14 are the same as the size and shape of the titanium 16. Therefore, germanium 14 is identical in shape and size of titanium 16 to enhance the appearance of the personal item.

Hologram element 12 is configured as a hologram chip disposed in the respective indention 22. A protective cover 28 is provided to cover at the respective indention 22 to seal and enclose the hologram element 12 in the respective indention 22, so that the hologram element 12 is hidden inside the body 10 in an invisible way. The hologram element 12 can be kept in tact to prolong the life span thereof. It is preferred that the protective cover 28 is made of germanium 14 or titanium 16. It is appreciated that the protective cover 28 can be made of the same material of the body 20 to form a unit body of the body 20.

Hologram element 12 contains frequency which can maintain the magnetic balance. It can continue to provide external magnetic energy to solve the problem of depletion of Earth's magnetic field. It can produce a subtle circular magnetic field flows through the human body, so that the electromagnetic flow back into balance, while making the human cells to restore the appropriate charge, to promote natural healing. It can improve blood circulation, increase metabolism, and prevent adult diseases.

Germanium 14 is a trace mineral that has been called one of the greatest new developments in the nutritional treatment of cancer. Organic germanium, also called Germanium-132 or Ge-Oxy 132, is preferred to be used and has been shown to protect the body from cancerous cell and tumor growth by strengthening the immune system. Germanium 14 may be a potential treatment for cancer as well as other degenerative diseases associated with aging and free radical damages.

Organic germanium 14 helps increases oxygen in the body, which has been shown to retard the growth of cancer cells and may even help return those cells to their normal state. Researchers discovered that cancer cells cannot survive in an oxygen-rich environment. Organic germanium carries oxygen across cellular membranes into the cells, which helps fight diseases caused by insufficient cellular oxygenation, or oxidative free-radical damage. One study of T-cells, B-lymphocytes, and natural killer cell activity, while increasing the number of anti-body-forming cells, there is no side effects founded thus far. Research suggests that germanium may help treat cancer of the lungs, bladder, larynx, and breast as well as depression, asthma, arthritis, heavy metal poisoning, sinus infection, diabetes, hypertension, heart disease, neuralgia, leukemia, and cirrhosis.

Titanium 16 is thought to attract oxygen. When held against the skin in form of a bracelet, titanium is believed to pull that oxygen into the area that is inflamed or is in pain. The excess oxygen relieves the pain and inflammation.

The magnetized titanium 16 can hold a positive charge. In alternative medicine, pain is said to have a negative charge. Magnetized titanium 16 can relieve pain by neutralizing the negative charge through the positive charge.

Unlike many other metals used in medicine, titanium 16 is compatible with the human body in that it can be used without fear or rejection by the body. If an implant is rejected, medical problems arise, the least of which being infection. The implant must be removed and replaced at immense cost to the patient. Titanium is common and known to overcome this obstacle and can remain inside the body for many years. Titanium can be carried without causing the carrier infection, rashes, or increased pain and swelling.

Pain is caused by an accumulation of toxins in specific parts of the body. Magnets have been used for centuries to relieve pain. Clinical studies and testimonials have influenced that recent popularity of titanium jewelry for pain relief as an alternative to oral medication. It works because magnetic energy stimulates the body to increase the flow of blood and oxygen. This not only relieves pain but reduces inflammation. It is especially useful for those with arthritis or sports injuries. This energy brings more flesh oxygen-filled blood to the affected area, which helps to flush away lactic acid.

Those with carpal tunnel syndrome or other repetitive injuries can heal faster by wearing titanium jewelry since it stimulates the nerves in the affected part of the body to promote healing. It also attracts iron in the red blood cells toward the affected area. Experts have discovered that the magnetic force of titanium can increase blood flow in the body by as much as 300 percent. The magnetic force from titanium can ensure vital energy is always available. Studies have shown that a continual and adequate magnetic energy can help the body achieve the balance it needs for the central nervous system to function correctly.

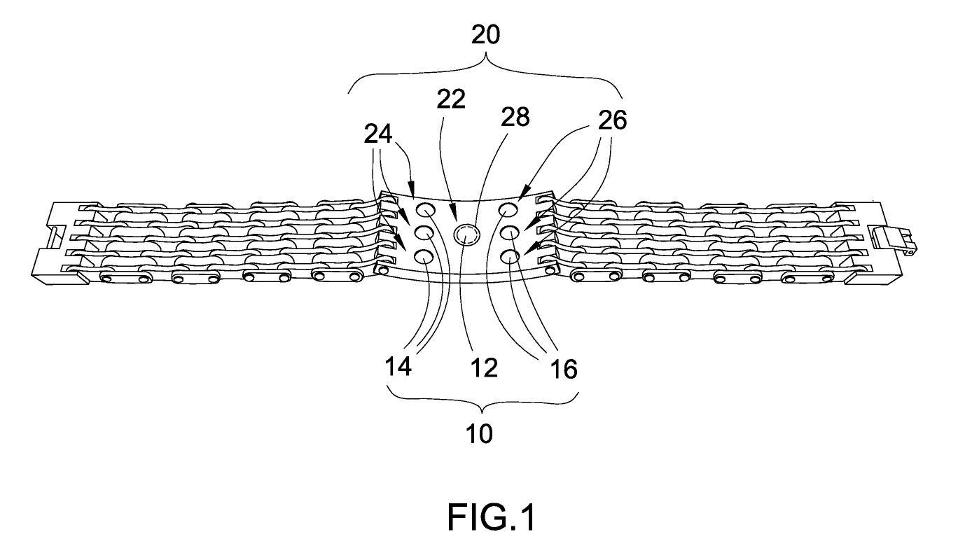

With regard to FIG. 1, the body 20 is embodied as jewelry such as a bracelet, wherein a bracelet body 20 thereof adapted to be worn by the user. The bracelet body 20 has two fastening ends detachably fastening with each other, and a bracelet platform 21 defined between the two fastening ends. It is preferred that the bracelet body 20 is made of stainless steel. Alternatively, the bracelet body 20 can made of precious metals such as titanium, platinum, silver, gold, etc. The energy elements 12, 14, and 16 are embedded at the inner surface of the bracelet platform 21 of the bracelet body 20, wherein the energy elements 12, 14, and 16 are irreplaceable.

The indentions 22, 24, 26 are indent on the inner surface of the bracelet platform 21 of the bracelet body 20. The hologram element 12, germanium 14, and titanium 16 are received in the indentions 22, 24, 26 respectively. Therefore, the hologram element 12, germanium 14, and titanium 16 are embedded in the inner surface of the bracelet platform 21 of the bracelet body 20 so that the energy elements 12, 14, 16 are located close to the user's skin when the bracelet body 20 is worn by the user. The protective cover 28 is provided to cover at the respective indention 22 to seal and enclose the hologram element 12 in the respective indention 22, so that the hologram element 12 is embedded and sealed at the inner surface of the bracelet platform 21 of the bracelet body 20 in an invisible way. It is noted that the hologram element 12 is sealed within the waterproof sleeve 18 and is received in the respective indentions 22. It is preferred that the protective cover 28 is made of germanium 14 or titanium 16. It is appreciated that the protective cover 28 can be made of stainless steel to form a unite body of the bracelet body 20. The protective cover 28 also can be made of precious metals such as titanium, platinum, silver, gold, etc.

It is appreciated that the bracelet body 20 can be configured as a watch body by providing a timepiece at the outer surface of the watch body and the energy elements 12, 14, and 16 at the inner surface of the watch body.

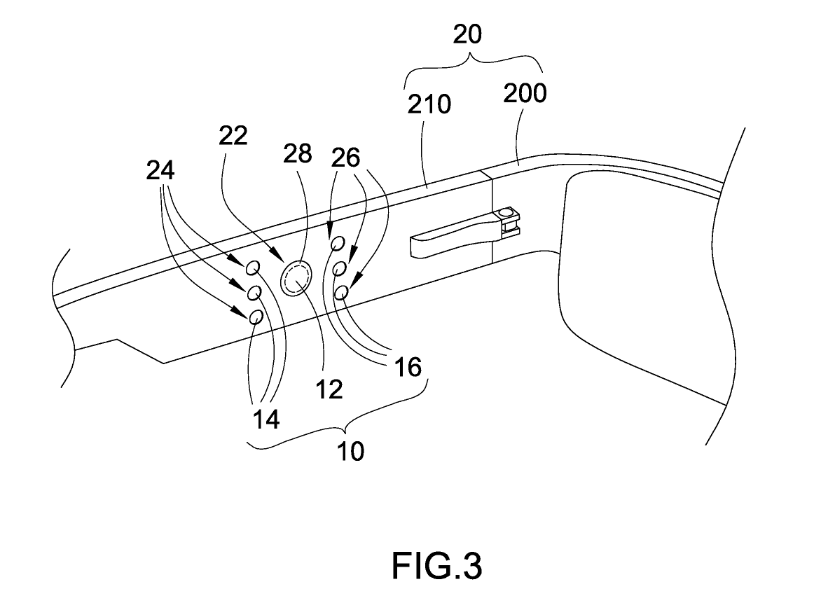

With regard to FIG. 3, the body 20 is embodied as a headwear such as a pair of spectacles. The spectacles generally comprise two lenses and a spectacle frame 200, i.e. the body 20, which comprises two temple arms 210 extended from two outer peripheral sides of the lenses respectively. Each temple arm 210 can be made of plastic or metal. The energy elements 12, 14, and 16 are embedded at the inner surface of the temple arms 210 of the spectacle frame 200, wherein the energy elements 12, 14, and 16 are irreplaceable. It is preferred that the energy elements 12, 14, and 16 are embedded at the inner surfaces of two temple arms 210 of the spectacle frame 200.

The indentions 22, 24, 26 are indent on the inner surface of at least one of the temple arms 210 of the spectacle frame 200. The hologram element 12, germanium 14, and titanium 16 are received in the indentions 22, 24, 26 respectively. Therefore, the hologram element 12, germanium 14, and titanium 16 are embedded in the inner surface of the temple arm 210 of the spectacle frame 200 so that the energy elements 12, 14, 16 are located close to the user's skin when the spectacle frame 200 is worn by the user. The protective cover 28 is provided to cover at the respective indention 22 to seal and enclose the hologram element 12 in the respective indention 22, so that the hologram element 12 is embedded and sealed at the inner surface of the temple arm 210 of the spectacle frame 200 in an invisible way. It is noted that the hologram element 12 is sealed within the waterproof sleeve 18 and is received in the respective indentions 22. It is preferred that the protective cover 28 is made of germanium 14 or titanium 16. It is appreciated that the protective cover 28 can be made of same material of the temple arm 210 to form a unite body of the spectacle frame 200.

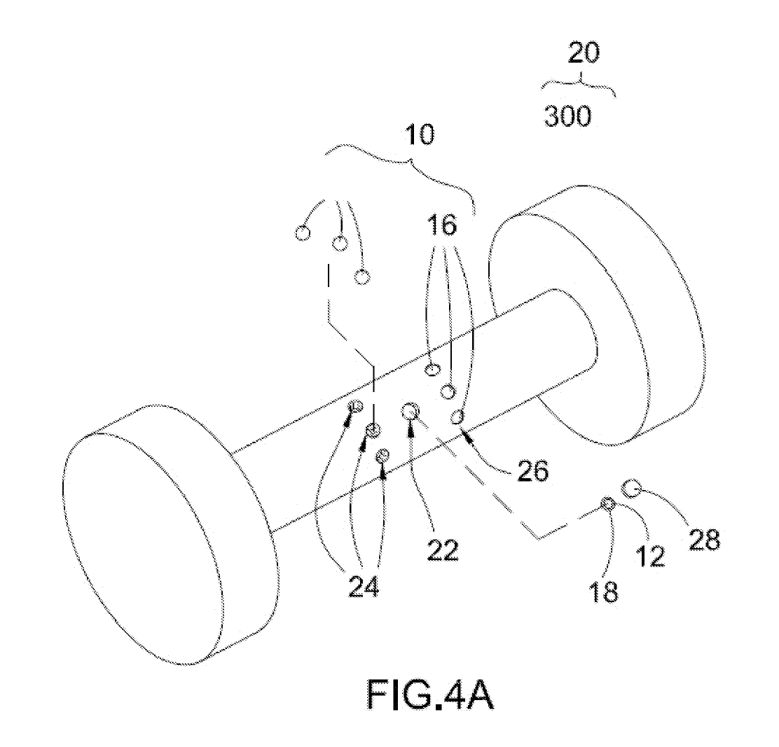

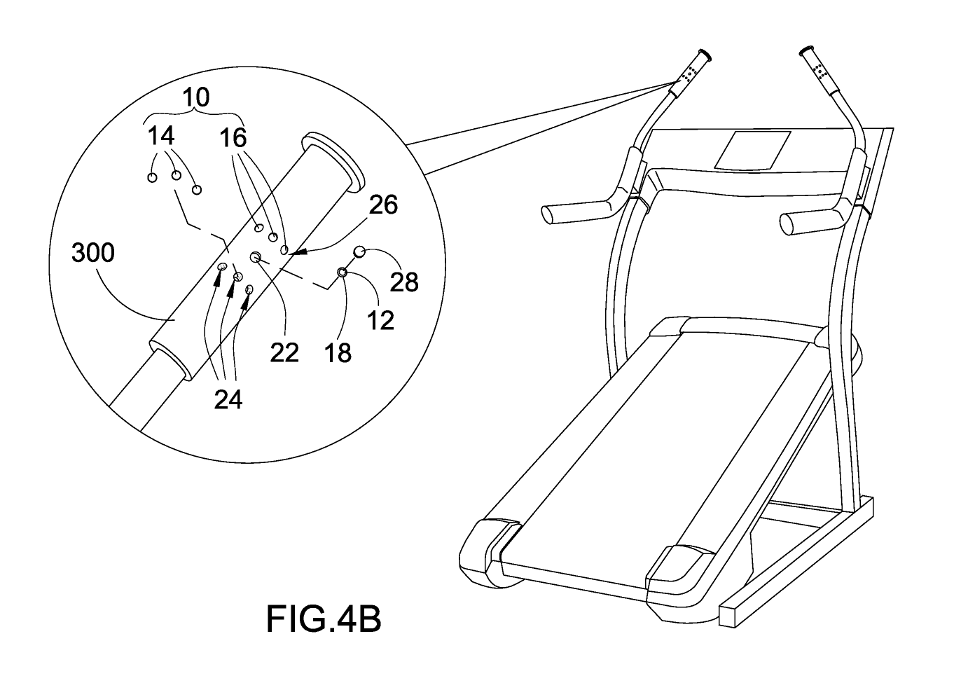

With regard to FIGS. 4A and 4B, the body 20 is embodied as a fitness gear, such as a dumbbell and treadmill respectively, wherein the energy elements 12, 14, and 16 are provided at a handle portion 300 (i.e. the body 20) of the fitness gear,

In FIG. 4A, the dumbbell generally comprises two weight members, wherein the handle portion 300 of the dumbbell, i.e. the body 20, extended between the weight members. The energy elements 12, 14, and 16 are embedded at the outer surface of the handle portion 300, wherein the energy elements 12, 14, and 16 are irreplaceable.

The indentions 22, 24, 26 are indent on the outer surface of the handle portion 300. The hologram element 12, germanium 14, and titanium 16 are received in the indentions 22, 24, 26 respectively. Therefore, the hologram element 12, germanium 14, and titanium 16 are embedded in the outer surface of the handle portion 300 so that the energy elements 12, 14, 16 are located close to the user's skin when the handle portion 300 of the dumbbell is carried by the user. The protective cover 28 is provided to cover at the respective indention 22 to seal and enclose the hologram element 12 in the respective indention 22, so that the hologram element 12 is embedded and sealed at the outer surface of the handle portion 300 in an invisible way. It is noted that the hologram element 12 is sealed within the waterproof sleeve 18 and is received in the respective indentions 22. It is preferred that the protective cover 28 is made of germanium 14 or titanium 16. It is appreciated that the protective cover 28 can be made of same material of the handle portion 300 to form a unite body of the handle portion 300.

With regard to FIG. 4B, the treadmill generally comprises an endless belt on which a user can run or jog, wherein the handle portion 300 of the treadmill, i.e. the body 20, is provided for the user to grip thereat. The energy elements 12, 14, and 16 are embedded at the outer surface of the handle portion 300, wherein the energy elements 12, 14, and 16 are irreplaceable.

The indentions 22, 24, 26 are indent on the outer surface of the handle portion 300. The hologram element 12, germanium 14, and titanium 16 are received in the indentions 22, 24, 26 respectively. Therefore, the hologram element 12, germanium 14, and titanium 16 are embedded in the outer surface of the handle portion 300 so that the energy elements 12, 14, 16 are located close to the user's skin when the handle portion 300 of the treadmill is gripped by the user. The protective cover 28 is provided to cover at the respective indention 22 to seal and enclose the hologram element 12 in the respective indention 22, so that the hologram element 12 is embedded and sealed at the outer surface of the handle portion 300 in an invisible way. It is noted that the hologram element 12 is sealed within the waterproof sleeve 18 and is received in the respective indentions 22. It is preferred that the protective cover 28 is made of germanium 14 or titanium 16. It is appreciated that the protective cover 28 can be made of same material of the handle portion 300 to form a unite body of the handle portion 300.

It is appreciated that the body 20 can be configured as other fitness gears such as gym equipment, wherein the energy elements 12, 14, and 16 are embedded at the outer surface of the desired location of the gym equipment.

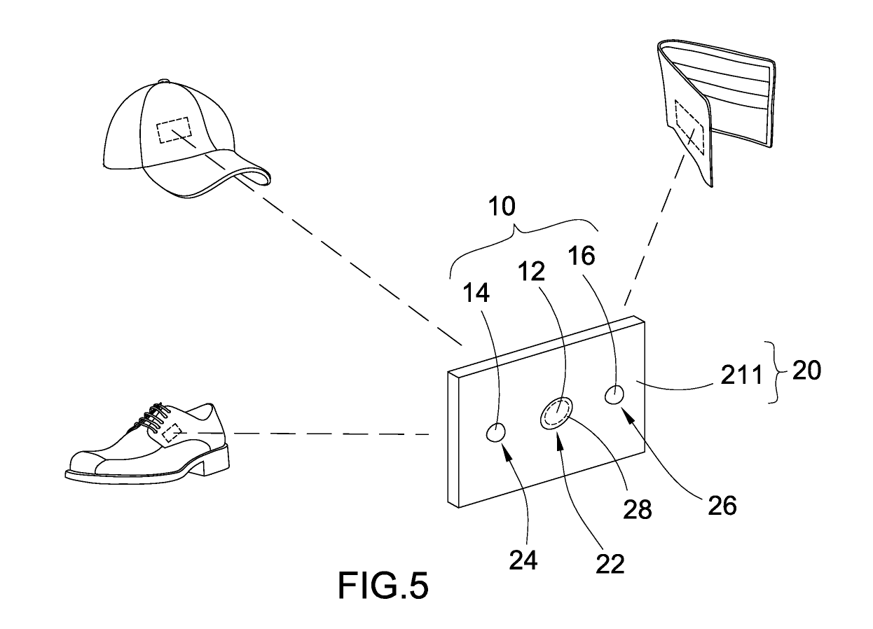

With regard to FIG. 5, the body 20 comprises an attachment platform 211 which can attach to any desired surface of the personal item such as cap, wallet, handbag, backpack, clothing, headwear, footwear, passport holder, key chain, or sporting equipment. It is preferred that the attachment platform 211 is made of stainless steel. Alternatively, the attachment platform 211 can be made of precious metals such as titanium, platinum, silver, gold, etc. The energy elements 12, 14, and 16 are embedded at the outer surface of the attachment platform 211 of the body 20, wherein the energy elements 12, 14, and 16 are irreplaceable.

The indentions 22, 24, 26 are indent on the outer surface of the attachment platform 211 of the body 20. The hologram element 12, germanium 14, and titanium 16 are received in the indentions 22, 24, 26 respectively. Therefore, the hologram element 12, germanium 14, and titanium 16 are embedded in the outer surface of the attachment platform 211 of the body 20 so that the energy elements 12, 14, 16 are located close to the user's skin when the body 20 is worn or carried by the user. The protective cover 28 is provided to cover at the respective indention 22 to seal and enclose the hologram element 12 in the respective indention 22, so that the hologram element 12 is embedded and sealed at the outer surface of the attachment platform 211 of the body 20 in an invisible way. It is noted that the hologram element 12 is sealed within the waterproof sleeve 18 and is received in the respective indentions 22. It is preferred that the protective cover 28 is made of germanium 14 or titanium 16. It is appreciated that the protective cover 28 can be made of same material of the attachment platform 211 to form a unite body of the body 20.

It is appreciated that the attachment body 211 in FIG. 5 is only for illustrative purpose in which the attachment body 211 can be attached to the cap, shoes, or wallet. With the configuration of the attachment body 211, the energy elements 12, 14, 16 can be retained at any desired object worn or carried by the user in order to locate the energy elements 12, 14, 16 close to the user's body in order to react positively with the user's body energy field.

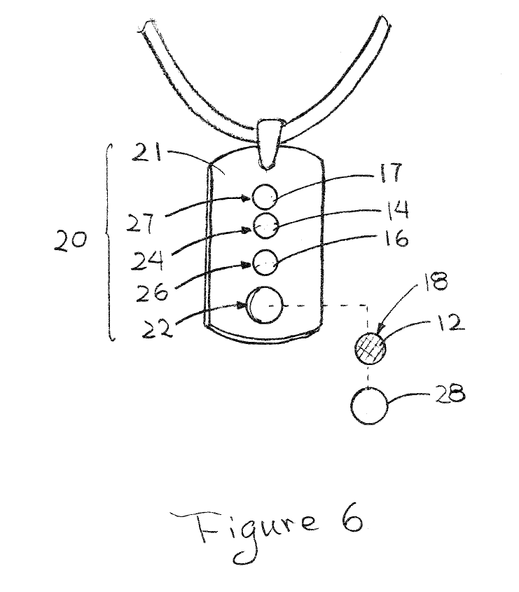

In FIG. 6, another embodiment shows the modification of the energy enhancement arrangement 10 which comprises a plurality of different energy elements 12, 14, and 16 selected from a group consisting of hologram element, germanium, titanium, and an element carrying negative ions. Hologram element, germanium, and titanium are assigned with a reference character 12, 14 and 16 in the figures respectively. The element with negative ions is assigned with a reference character 17.

Negative Ions are created naturally in nature by the energy associated with waterfalls, rolling surf and lightning. Negative ions are negatively charged particles form when enough energy acts on a molecule to eject an electron. This displaced electron then attaches itself to a nearby molecule, which then becomes a Negative Ion.

Direct Effects of Negative Ion on Human Health

Activate cells: through cell membrane, it promote exchange of calcium and natrium inside and outside membrane, and keep cell's activity.

Blood purification: through cell's activity, it promote cell metabolism, and excreted hazardous waste and toxic substances from body.

Resume fatigue: eliminate illness factors, quickly recover physical strength.

Stabilize botanic nerve system: influence botanic nerve system to obtain rirritable ability, adjust function of botanic nerve system.

Strengthen resistance capacity to disease: stabilize emotion, increase C-Bail globins content, improve quantity and quality of leukin, and boost leukin's phagocytic function to the bacteria.

Restrain tumor cell growing: influence the composition and distribution of charged particles in blood, and increase cell's activity.

Absorb and shield electromagnetic radiation. Improve allergic physique: adjust the balance of botanic nerve system, strengthen the resistance to allergen material.

The Main Performance of Negative Ion for Human Body

The influences for nervous system: enhance pallium function and brain activity, excite nerve, improve work efficiency, and improve sleep quality. Negative ion also can strength the intensity of oxidation process of brain tissue, and help brain tissue to obtain much oxygen. Influences to cardiovascular system: negative ion has obvious effects to vascular expansion, restrain cholesterol content of serum, purify blood, avoid lipid depositing on vascular wall, improve blood circulation, relieve artery vasospasm, and achieve to maintain steady blood pressure.

Negative ion has big benefits for improving cardiac function and myocardial nutrition, and is good for the rehabilitation of hypertension and vascular diseases patients.

Influences to blood system: studies proved that negative ion has function to slow blood and extend clotting time, increase the oxygen content in blood, and thus is beneficial to oxygen transmission, absorption and utilization.

To improve NK activity to damage cancer cell; restrain the happening and transfer of cancer cell.

To restrain colorless leukotriene that could cause allergy and angry state, could be used in beauty and skin treatment. For example, skin burns can be quickly cured by negative ion. Negative ion also has analgesic effect.

To prevent artery probation or peroxide lipid of liver, too much ions may cause oxidation of axunge, quick oxidation of axunge will accelerate body aging.

Remove skin waste, increase activity of the skin, has significant effect for beauty.

Accordingly, negative ion is closely related to human's all systems, that is, human health, therefore it is will play an obvious role in human health.

In accordance with the present invention, the element carrying the negative ions is a natural element selected from the group consisting of silicone, Tourmaline, and Volcanic rock.

With regard to FIG. 6, the body 20 is embodied as pendant necklace, wherein a pendant body 20 thereof adapted to be worn by the user. The pendant body 20 has a hanging loop adapted for being worn via a chain, and a pendant platform 21 as one of two flat surfaces. It is preferred that the pendant body 20 is made of stainless steel. Alternatively, the pendant body 20 can made of precious metals such as titanium, platinum, silver, gold, etc. The energy elements 12, 14, 16 and 17 are embedded at the one surface of the pendant platform 21 of the pendant body 20, wherein the energy elements 12, 14, 16, and 17 are irreplaceable.

The indentions 22, 24, 26, 27 are indent on the inner surface of the pendant platform 21 of the pendant body 20. The hologram element 12, germanium 14, titanium 16, and the element carrying negative ions 17 are received in the indentions 22, 24, 26, 27 respectively. Therefore, the hologram element 12, germanium 14, titanium 16, and the element carrying negative ions 17 are embedded in the inner surface of the pendant platform 21 of the pendant body 20 so that the energy elements 12, 14, 16, 17 are located close to the user's skin when the pendant body 20 is worn by the user. It is noted that the hologram element 12 is sealed within the waterproof sleeve 18 and is received in the respective indentions 22. The protective cover 28 is provided to cover at the respective indention 22 to seal and enclose the hologram element 12 in the respective indention 22, so that the hologram element 12 is embedded and sealed at the inner surface of the pendant platform 21 of the pendant body 20 in an invisible way. It is preferred that the protective cover 28 is made of germanium 14 or titanium 16 to reduce one of the indention spaces on the pendant body 20. It is appreciated that the protective cover 28 can be made of the same material of the pendant body 20 to form a unit body of the pendant body 20. The pendant body 20 is made of material selected from a group consisting of stainless steel, titanium, platinum, silver, and gold.

While the embodiments and alternatives of the present invention have been shown and described, it will be apparent to one skilled in the art that various other changes and modifications can be made without departing from the spirit and scope of the present invention.

What is claimed is:

1. A personal item, comprising:
an energy enhancement arrangement which comprises a plurality of different energy elements, each of said energy elements adapted for generating a particular energy which helps rebalance and restore natural energy pathways of a user, wherein said energy elements are hologram element, germanium, and titanium; and
a body adapted for being carried by said user, wherein at least two of said different energy elements are held by said body, whereby, when said user carries said body, said energy elements are held close enough to said user in order to react positively with said user's energy field.

2. The personal item of claim 1 further comprising an element carrying negative ions as one of said energy elements being held by said body.

3. The personal item of claim 1 wherein said hologram element is a hologram chip and said germanium is organic germanium.

4. The personal item of claim 2 wherein said hologram element is a hologram chip and said germanium is organic germanium.

5. The personal item of claim 1 wherein said energy elements are embedded into said body so that said energy elements are irreplaceable to said body.

6. The personal item of claim 4 wherein said energy elements are embedded into said body so that said energy elements are irreplaceable to said body.

7. The personal item of claim 1 wherein said body has a corresponding number of spaced apart indentions formed at a surface of said body to receive said energy elements respectively.

8. The personal item of claim 6 wherein said body has a corresponding number of spaced apart indentions formed at a surface of said body to receive said energy elements respectively.

9. The personal item of claim 7 wherein said indentions are formed at an inner surface of said body so that said energy elements are located close to said user's skin when said body is worn by said user.

10. The personal item of claim 8 wherein said indentions are formed at an inner surface of said body so that said energy elements are located close to said user's skin when said body is worn by said user.

11. The personal item of claim 7 wherein said body further comprises a protective cover covering at one of said indentions to seal and enclose said hologram element in said respective indention, so that said hologram element is hidden inside said body in an invisible way.

12. The personal item of claim 10 wherein said body further comprises a protective cover covering at one of said indentions to seal and enclose said hologram element in said respective indention, so that said hologram element is hidden inside said body in an invisible way.

13. The personal item of claim 11 wherein said protective cover is made of said germanium or said titanium.

14. The personal item of claim 12 wherein said protective cover is made of said germanium or said titanium.

15. The personal item of claim 2 wherein said element carrying said negative ions is a natural element selected from the group consisting of silicone, Tourmaline, and Volcanic rock.

16. The personal item of claim 14 wherein said element carrying said negative ions is a natural element selected from the group consisting of silicone, Tourmaline, and Volcanic rock.

17. The personal item of claim 1 wherein said body is selected from a group consisting of bracelet, jewelry, watches, clothing, headwear, footwear, eyewear, key chain, purse, wallet, belt, sporting goods, and pendant.

18. The personal item of claim 16 wherein said body is selected from a group consisting of bracelet, jewelry, watches, clothing, headwear, footwear, eyewear, key chain, purse, wallet, belt, sporting goods, and pendant.

19. The personal item of claim 1 wherein said body is made of material selected from a group consisting of stainless steel, titanium, platinum, silver, and gold.

20. The personal item of claim 18 wherein said body is made of material selected from a group consisting of stainless steel, titanium, platinum, silver, and gold.
